(19) United States
(12) Patent Application Publication
(10) Pub. No.: US 2023/0405081 A1
(43) Pub. Date: Dec. 21, 2023

(54) PHARMACEUTICAL COMPOSITION COMPRISING A PEPTIDE AS ACTIVE INGREDIENT

(71) Applicant: INTRACT PHARMA LIMITED, London (GB)

(72) Inventor: Vipul YADAV, London (GB)

(73) Assignee: INTRACT PHARMA LIMITED, London, EN (GB)

(21) Appl. No.: 18/252,438

(22) PCT Filed: Nov. 11, 2021
(86) PCT No.: PCT/EP2021/081340

§ 371 Date: May 10, 2023

(30) Foreign Application Priority Data
Nov. 12, 2020 (GB) 2017863.8

(51) Int. Cl. A61K 38/095; A61K 9/00; A61K 9/28

(52) U.S. Cl. CPC A61K 38/095; A61K 9/0053; A61K 9/0031; A61K 9/2866; A61K 9/2846

(57) ABSTRACT

A pharmaceutical composition which comprises (a) as active ingredient a peptide, and (b) an amino acid, dipeptide or a combination thereof, which act as a stabilising agent is described.

### FIELD OF THE INVENTION

This invention relates to novel compositions and their use.

### BACKGROUND

Biopharmaceuticals, and particularly peptides, are an increasingly important class of pharmaceuticals, and many therapeutic uses have been proposed and implemented. However, delivery of biopharmaceuticals to the desired target continues to be a major challenge in pharmaceutical industry as well as an unmet medical need for several diseases. Specifically, oral or rectal administration can be ineffective, because the molecules are highly unstable in the presence of gastric and intestinal fluids. Hence for this reason, intravenous or subcutaneous delivery remains the most feasible option for delivery of proteins and peptides. However, such delivery is not usually the most convenient method of administration of a drug to a patient, and issues include pain at the site of injection, compliance, and frequent hospital visits for intravenous infusions. There can also be other major disadvantages of intravenous or subcutaneous delivery, and for some applications serious systemic adverse effects. High systemic exposure can also lead to a decline in efficacy with repeated administration due to formation of anti-drug antibodies.

There is a distinct unmet medical need for safer, more effective, patient-friendly biotherapeutic approaches which avoid the issues inherent in the use of injected drugs.

For some drugs, targeting of the drug to the colon has been utilised as a means of achieving local therapy or systemic treatment. For example, WO 2007/122374 describes compositions having a delayed release coating which can be used to target release of a drug from a core to the intestine, particularly the colon. The colon is susceptible to a number of disease states, including IBD, irritable bowel syndrome (IBS), constipation, diarrhoea, infection and carcinoma. IBD is a chronic, medically incurable condition involving inflammation of the gut.

There are two main forms of IBD: ulcerative colitis (UC), which typically begins in the descending colon and rectum and may extend continuously to involve the entire colon (pancolitis), and Crohn's disease (CD), which most commonly involves the terminal small intestine and ascending colon. UC usually affects only the epithelial layer of the bowel wall, while CD may affect all layers of the intestinal wall. A number of biological therapies are currently available for the treatment of IBD, but because of the stability problems discussed above, none is currently available as an oral treatment.

There remains an unmet need for a method of stabilising peptides in the presence of luminal fluid found in the lower gastrointestinal tract, such as the small intestine, and/or the colon. Specifically, there remains a need for a method of stabilising peptides sufficiently for them to be delivered to the ileum and/or the colon by rectal or, especially, oral administration.

The present inventors have found that it is possible to reduce breakdown of peptide molecules by co-administration of single amino acids, di-peptides, or a combination of amino acids and dipeptides.

### SUMMARY OF THE INVENTION

Accordingly, the present invention provides a pharmaceutical composition which comprises (a) as active ingredient a peptide, and (b) an amino acid, dipeptide or a combination thereof.

The amino acid and/or dipeptide act as a stabilising agent. Preferably the pharmaceutical composition is suitable for administration via the intestinal tract.

The peptide may be a linear or a cyclic peptide. Preferably the peptide is a cyclic peptide. More preferably the peptide is desmopressin or oxytocin or a variant thereof.

The composition of the invention may be in liquid or in solid or in semi-solid form, preferably in a form suitable for rectal or, especially, oral administration. The compositions may be in a solid form suitable for oral administration, said composition having an enteric coating. Most preferably it is in a solid or semi-solid form suitable for oral administration, and adapted for selective release of the peptide in the lower gastrointestinal tract, in particular the ileum and/or the colon.

The composition may comprise a solid dosage form with a core and a coating for the core, the core comprising as an active ingredient a peptide, and the amino acid, dipeptide or a combination thereof; and the coating comprising a mixture of a digestible polysaccharide and a film-forming material which has a solubility threshold at pH 6.0 or above.

The composition may be an orally administrable pharmaceutical composition comprising (a) as active ingredient a peptide, and (b) an amino acid, dipeptide or a combination thereof.

The invention also provides a solid dosage form for oral administration comprising a core comprising (a) as active ingredient a peptide, and (b) an amino acid, dipeptide or a combination thereof; and a delayed release coating for the core.

The invention also provides a rectally administrable pharmaceutical composition comprising (a) as active ingredient a peptide, and (b) an amino acid, dipeptide or a combination thereof.

The invention also provides an enema formulation comprising (a) as active ingredient a peptide, and (b) an amino acid, dipeptide or a combination thereof.

The present invention further provides a pharmaceutical composition according to the invention for use in therapy. Further, there is also provided a pharmaceutical composition according to the invention for use in the treatment or prevention of disease or conditions selected from an inflammatory bowel disease; irritable bowel syndrome; constipation; diarrhoea; infection; or cancer. Preferably the pharmaceutical composition is a pharmaceutical composition of the invention.

The invention further provides a method of treating or preventing a disease or condition in a subject which comprises administering to the subject a pharmaceutical composition according to the invention. Preferably, the disease or condition is selected from inflammatory bowel disease; irritable bowel syndrome; constipation; diarrhoea; infection; autoimmune disease or cancer. Preferably the pharmaceutical composition is a pharmaceutical composition of the invention.

The present invention further provides a method of stabilising a peptide in the presence of intestinal fluid, which comprises delivering a peptide as an active ingredient and an amino acid, dipeptide or a combination thereof.

The invention also provides the use of an amino acid, dipeptide or a combination thereof for the stabilisation of a peptide which has been administered as a pharmaceutical composition and delivered to the lower gastro-intestinal tract. Preferably the pharmaceutical composition is a pharmaceutical composition of the invention.

### DETAILED DESCRIPTION OF THE INVENTION

As used herein “lower gastrointestinal tract” refers to gastrointestinal tract after the stomach. This includes the small intestine and large intestine. The small intestine is made up of the duodenum, jejunum and ileum, while the large intestine is also known as the colon. Preferably, the stabilising agents reduce degradation of the peptide in the ileum and/or colon.

The Peptide

The peptide may be any peptide having a beneficial therapeutic effect towards human or animals.

Peptides are chains of amino acid monomers joined together by amide bonds. Peptides can be made up of two or more amino acid monomers. Preferably the peptides contain less than 50 amino acid monomers, preferably 2-50 amino acid monomers, more preferably 2-10 amino acid monomers, 5-20 amino acid monomers, 15-40 amino acid monomers or 18-30 amino acid monomers. Preferably the peptides have a molecular weight up to 5 kDa, more preferably between 1-2.5 kDa. Suitable peptides include linear, branched and cyclic peptides with disulphide bridges. Cyclic peptides may include 1-5 disulfide bridges, preferably 1-3 disulfide bridges. Preferably the peptide is a cyclic peptide.

The peptides are generally made from naturally occurring L form amino acids. Variant peptides may contain one or more D-form and/or non-natural amino acids, such as those with modified side groups. Preferably the variant peptide has one, two, three, four or five D form amino acids. Preferably in a variant peptide, an L form amino acid is replaced by the equivalent D form amino acid, or the equivalent amino acid with a modified side group. In addition variant forms of the peptide may be linear or cyclic versions of normally cyclic or linear peptides respectively.

One preferred peptide is oxytocin and variants thereof. Oxytocin has the amino acid sequence Cys-Tyr-Ile-Gln-Asn-Cyc-Pro-Leu-Gly, and usually contains a sulfide bridge between the cysteine residues. One preferred variant, having one D amino acid (Leu), has the following structure

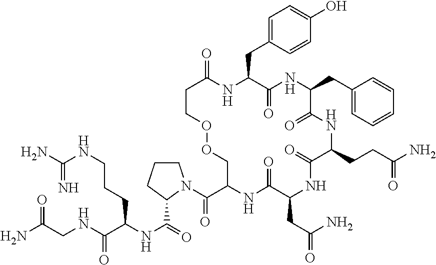 [SMILES: N=C(N)NCCC[C@@H](NC(=O)[C@@H]1CCCN1C(=O)C1COOCCC(=O)N[C@@H](Cc2ccc(O)cc2)C(=O)N[C@@H](Cc2ccccc2)C(=O)N[C@@H](CCC(N)=O)C(=O)N[C@@H](CC(N)=O)C(=O)N1)C(=O)NCC(N)=O]

In another variant the cyclic peptide is linearised.

One preferred peptide is Desmopressin (D-amino D arginine vasopressin), which is a cyclic peptide containing 9 amino acids, or a variant thereof. The structure of desmopressin is provided below:

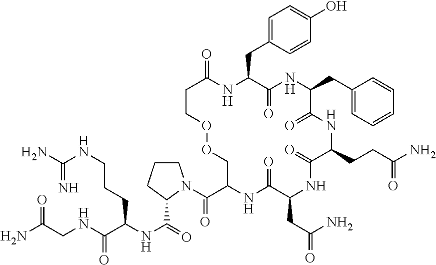 [SMILES: N=C(N)NCCC[C@@H](NC(=O)[C@@H]1CCCN1C(=O)C1COOCCC(=O)N[C@@H](Cc2ccc(O)cc2)C(=O)N[C@@H](Cc2ccccc2)C(=O)N[C@@H](CCC(N)=O)C(=O)N[C@@H](CC(N)=O)C(=O)N1)C(=O)NCC(N)=O]

Stabilising Agents

The amino acid, dipeptide or a combination thereof act as stabilising agents to help to maintain the intact peptide in the lower gastrointestinal tract. Methods for assessing the stability of the peptide in gastrointestinal fluid are described in the Examples.

The amino acid, dipeptide or a combination thereof cause at least 5% of the peptide present to remain intact after 4 hours in the gastrointestinal fluid. Preferably at least 10%, 20%, 30%, 40%, 50%, 60%, 70%, 75%, 80%, 90% 95% or more of the peptide remains intact.

Amino Acids

Amino acids are organic compounds containing amine (—NH₂) and carboxyl (—COOH) groups, along with a side chain which is specific to each amino acid. As used herein, the term “amino acid”, when used in reference to a stabilising agent includes the 21 proteinogenic L-amino acids found in eukaryotes (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine) as well as D-form and non-natural amino acids. The term also includes include non-naturally occurring amino acids, such as those with modified side groups.

Preferably the amino acid is selected from Phenylalanine, Tyrosine, Glycine, Arginine, preferably L-arginine or combinations thereof. Preferably the amino acid is selected from Phenylalanine, Tyrosine, and Glycine, or combinations thereof. Preferably the amino acid is Arginine, preferably L-arginine. The composition may comprise more than one amino acid, such as 2, 3, or 4 amino acids. Preferably the composition comprises two amino acids.

Preferably the composition comprises glycine and phenylalanine or tyrosine and phenylalanine.

Dipeptides

Dipeptides are molecules comprising 2 amino-acids linked by a peptide bond. The amino acids can be naturally occurring or chemically modified versions. Examples of di-peptides are diglycine, Gly-Phe, and Tyr-Phe as described below.

Diglycine

Diglycine is a dipeptide which consists of two glycine amino acids linked together through a peptide bond. It has the following structure.

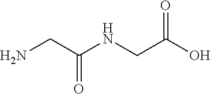

Gly-Phe

Gly-Phe is a dipeptide which consists of one glycine molecule linked together with one phenylalanine molecule through a peptide bond. It has the following structure.

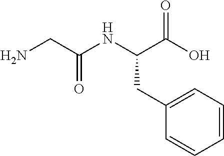 [SMILES: NCC(=O)N[C@@H](Cc1ccccc1)C(=O)O]

Tyr-Phe

Tyr-Phe is a dipeptide which consists of one tyrosine molecule linked together with one phenylalanine molecule through a peptide bond.

The ratio of peptide to amino acid and/or dipeptide is in the range 1:3 to 1:20. The preferred ratio is in the range 1:4 to 1:10. The preferred ratio of peptide:glycine is in range 1:3 to 1:5, more preferably 1:4. The preferred ratio of peptide:tyrosine is in range 1:6 to 1:8, more preferably 1:7. The preferred ratio of peptide:phenylalanine is in range 1:4 to 1:8, more preferably 1:6.6. The preferred ratio of peptide:arginine is in range 1:5 to 1:15, more preferably 1:10.

Pharmaceutical Formulations

The pharmaceutical compositions according to the invention are preferably in solid or semi-solid form, and preferably they are suitable for oral or rectal administration.

The composition may also be in the form of a lotion, cream, foam, emulsion or gel. Such formulations may be prepared by a number of known methods established in the art. For example, the peptide and the amino acid, dipeptide or a combination thereof may be admixed together, optionally together with other excipients required in the dosage form.

Pharmaceutical compositions in the present invention that are suitable for oral administration may be presented either in the form of tablets, capsules, mini-tablets, pellets, powders, granules, microparticles, nanoparticles or hydrogels.

Compositions of the present invention suitable for oral administration may be presented as discrete units such as capsules, tablets, mini-tablets, or pellets, or as powders, granules or crystals. In a solid composition, the minimum diameter of each particle is typically at least 10⁻⁴ m, usually at least 5×10⁻⁴ m and, preferably at least 10⁻³ m. The maximum diameter is usually no more than 30 mm, typically no more than 20 mm and, preferably, no more than 10 mm. In preferred embodiments, the particle has a diameter from about 0.2 mm to about 15 mm, preferably from about 1 mm to about 4 mm (e.g. for pellets or mini-tablets) or from about 6 mm to about 12 mm (e.g. for certain tablets or capsules). The term “diameter” refers to the largest linear dimension through the particle.

As well as the required amino acid and/or dipeptide, which act as a stabilising agent, compositions according to the invention may of course contain any further conventional excipients as required such as binders, extenders, disintegrants, diluents and lubricants.

Excipients used in solid forms include for example, microcrystalline cellulose, dicalcium phosphate, starch, magnesium stearate, calcium sulfate, sorbitol, glucose and/or lactose and/or other excipients, binders, extenders, disintegrants, diluents and lubricants known in the art. Suitable binders include starch, gelatine, natural sugars such as glucose or beta-lactose, corn sweeteners, natural and synthetic gums such as acacia, tragacanth or sodium alginate, carboxymethylcellulose, polyethylene glycol, waxes and the like. Disintegrators include without limitation starch, methylcellulose, agar, bentonite, xanthan gum and the like. Fast dissolving diluents include mannitol, lactose, sucrose and/or cyclodextrins. Lubricants, glidants, flavours, colouring agents and stabilizers may also be added for ease of fabrication and use. Lubricants include sodium oleate, sodium stearate, magnesium stearate, sodium benzoate, sodium acetate, sodium chloride.

A tablet may be made by compression or moulding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Moulded tablets may be made by moulding in a suitable machine a mixture of the powdered compound moistened with an inert liquid diluent. The tablets may optionally be coated or scored and may be formulated so as to provide slow, delayed or controlled release of the antibody. Preferred examples of coatings are given below.

Capsules may have solid, semi-solid or non-solid contents. Exemplary contents for capsules may include suspensions which can contain, for example, microcrystalline cellulose for imparting bulk, alginic acid or sodium alginate as a suspending agent, and methylcellulose as a viscosity enhancer, as well as any of the solid or semi-solid forms above.

Formulations for rectal administration may be presented as a suppository with the usual carriers such as cocoa butter, synthetic glyceride esters or polyethylene glycol. Such carriers are typically solid at ordinary room temperatures (up to 25° C.), but liquefy and/or dissolve in the rectal cavity to release the drug.

Compositions may also take the form of an enema formulation such as a liquid or foam enema which is rectally administered to the lower colon. The enema formulations typically comprise the peptide, together with an amino acid, dipeptide or combinations thereof, dissolved or dispersed in a suitable flowable carrier vehicle, such as deionised and/or distilled water. The formulation can be thickened with one or more thickeners. They may also contain a buffer, and can also comprise an effective amount of a lubricant such as a natural or synthetic fat or oil, e.g. a tris-fatty acid glycerate or lecithin. Non-toxic non-ionic surfactants can also be included as wetting agents and dispersants. A buffer is preferably added to the liquid or foam enema to stabilise the pH. The pH is preferably 3.5 to 7.5, especially 6.5 to 7.5.

Unit doses of enema formulations can be administered from pre-filled bags or syringes. In the case of a pressurised enema formulation the carrier vehicle may also comprise an effective amount of a foaming agent such as n-butane, propane or i-butane, or the foaming agent/propellant could be held separately from the composition such as in a bag-in-bag or bag-in-can system as described in WO-A-9603115 (incorporated herein by reference). Enema foams may also comprise expanding agents and foam-stabilisers.

The volume of a liquid enema is typically 50-200 cm³, preferably about 100 cm³. The volume of a foam enema is typically 20 to 40 cm³. A suitable dosage of the amino acid, dipeptide or a combination thereof in the enema as administered is mM to 100 mM, preferably 10 mM to 75 mM, more preferably 40 mM to 50 mM.

Preferred unit dosage formulations are those containing an effective dose, or an appropriate fraction thereof, of the active ingredient. Release from certain formulations may also be sustained, if the composition contains suitable controlled-release excipients. However, in preferred formulations, release is pulsatile.

The compositions according to the invention will typically comprise a therapeutically effective amount of the peptide which may be from 0.01 wt % to 99 wt %, based on the total weight of the composition. The actual dosage would be determined by the skilled person using common general knowledge. However, by way of example, “low” dose formulations typically comprise no more than 20 wt % of the peptide, and preferably comprise from 1 wt % to 10 wt %, e.g. 5 wt %, of the peptide. “High” dose formulations typically comprise at least 40 wt % of the peptide, and preferably from 45 wt % to about 85 wt %, e.g. 50 wt % or 80 wt %.

The compositions according to the invention will typically comprise an effective amount of the amino acid, dipeptide or a combination thereof as stabilising agents. Typically, formulations comprise 0.01 wt % to 99 wt % of the amino acid, dipeptide or a combination thereof, based on the total weight of the composition The compositions may preferably comprise no more than 20 wt % of the amino acid, dipeptide or a combination thereof, and preferably comprise from 1 wt % to 10 wt %, e.g. 5 wt %, of the amino acid, dipeptide or a combination thereof. Alternatively, the compositions may comprise at least 40 wt % of the amino acid, dipeptide or a combination thereof, and preferably from 45 wt % to about 85 wt %, e.g. 50 wt % or 80 wt %.

Whilst the peptide may be used as the sole active ingredient in a composition according to the invention, it is also possible for the peptide to be used in combination with one or more further therapeutic agents. Thus, the invention also provides a composition according to the invention containing a further therapeutic agent in addition to the peptide. If desired, the composition according to the invention may be administered together with a further composition, by simultaneous, sequential or separate administration.

Except where the context requires otherwise, throughout this Specification and claims, any reference to a pharmaceutical composition in solid or semi-solid form should be understood to include individual solid or semi-solid particles or unit forms which are solid or semi-solid throughout, as well as those having a solid or semi-solid exterior and a non-solid, for example liquid or gel, interior. For example, a capsule may have liquid or gel contents.

Delivery to the Lower Gastrointestinal Tract

The composition according to the invention is adapted for delayed or selective release of the peptide in the lower gastrointestinal tract, in particular the ileum and/or, especially, the colon, suitably following rectal or, especially, oral administration. This may be accomplished by the use of particular coatings. The compositions of the invention may be delayed release oral (DRO) compositions. The DRO compositions pass through the stomach substantially unaltered and deliver the active ingredient to the lower gastrointestinal tract, typically the ileum and/or colon (i.e. the site of the diseased mucosa).

The compositions according to the invention may have an enteric coating. Enteric coatings protect the active ingredients in a composition from attack and degradation in the stomach, but dissolve and release to contents of the dosage form within the intestines, usually due to the change in pH. Suitable enteric coatings are well known in the art. The optimal coating for any particular formulation depends on the exact intended use, and coatings may be tailored to release the active ingredient in a particular region of the intestines, or at a particular time following ingestion. Such a formulation may if desired contain one or more intermediate layers between the active ingredient and the outer enteric coating. In this case, it is possible for a composition of the invention to release a portion of its contents at one particular region of the intestine, and a further portion of its contents in a second region of the intestine, such as the colon. Preferably, the composition of the present invention is in a solid or semi-solid form which comprises an enteric coating adapted to release the peptide in the colon. Useful enteric coatings are those which remain intact in the low pH environment of the stomach, but readily dissolve when the optimum pH for dissolution is reached. This can vary between pH 3 to 7.5, preferably 5 to 7, depending on the chemical composition of the coating. The thickness of the coating required will depend on the solubility of the coating and the intended site to be treated. Typically, the coating is 25 to 200 μm, especially 75 to 150 μm.

The composition of the invention is adapted for release of the active ingredient to the part of the lower gastrointestinal tract where the disease is prevalent. Typically the enteric coating should dissolve in the pH of the jejunum (about pH 5.5), ileum (about pH 6) and/or colon (pH 6-7) to that the majority of the peptide is released at the desired site.

WO 2007/122374 (the contents of which are incorporated herein by reference) describes compositions for selective release within the colon, and these form one preferred embodiment of the invention. Accordingly, the invention further provides a composition comprising a particle with a core and a coating for the core, the core comprising a peptide as active ingredient together with an amino acid, dipeptide or a combination thereof as a stabilising agent, the coating comprising a mixture of a mixture of a digestible polysaccharide and a film-forming material which has a solubility threshold at pH 6.0 or above, preferably pH 7 or above.

The digestible polysaccharide is susceptible to attack by intestinal bacteria. Preferably the digestible polysaccharide is selected from the group consisting of starch; amylose; amylopectin; chitosan; chondroitin sulfate; cyclodextrin; dextran; pullulan; carrageenan; scleroglucan; chitin; curdulan and levan.

For example, the polysaccharide may be starch, amylose or amylopectin.

The film-forming material is an enteric material which has a pH threshold which is the pH below which it is insoluble and at or above which it is soluble. The pH of the surrounding medium triggers dissolution of the second material. The normal pH of gastric juice is usually in the range of 1 to 3, while the pH of intestinal juice gradually increases from about 5.5 in the duodenum to about 7 to 8 in the colon. Thus, the second material, when used in a composition of the present invention, has a pH threshold of 6.0 or greater, especially 7 or greater.

The film-forming material is typically elected from an acrylate polymer, a cellulose polymer or a polyvinyl-based polymer. Examples of suitable cellulose polymers include cellulose acetate phthalate (“CAP”); cellulose acetate trimellitate (“CAT”); and hydropropylmethylcellulose acetate succinate. Examples of suitable polyvinyl-based polymers include polyvinyl acetate phthalate (“PVAP”). The film-forming material is preferably a co-polymer of a (meth)acrylic acid and a (meth)acrylic acid C₁₋₄ alkyl ester, for instance, a copolymer of methacrylic acid and methacrylic acid methyl ester. Such polymers include those available under the Trade Marks Eudragit L, Eudragit S and Eudragit FS. The use of Eudragit S as the film-forming material is particularly preferred.

In such compositions, multi-unit dosage forms comprising particles having a diameter of less than 3 mm are preferred. The “core” is usually a single solid body. The core may consist of the peptide together with the amino acid, dipeptide or a combination thereof. More usually, however, the core will comprise a mixture of the peptide and the amino acid, dipeptide or a combination thereof, and optionally one or more additional excipient. The core may for example include a filler or diluent material, e.g. lactose or cellulose material such as microcrystalline cellulose; a binder, e.g. polyvinylpyrrolidone (PVP); a disintegrant, e.g. croscarmellose sodium; and/or a lubricant, e.g. magnesium stearate. The core may be a compressed granulate comprising at least some of these materials.

Release from such compositions is delayed until the lower gastro-intestinal tract, in particular the ileum and/or the colon. Such compositions have application in a multi-phasic release composition comprising at least two pluralities of particles, e.g. coated pellets, in the same dosage form, e.g. a capsule, in which the particles of one plurality are differentiated from the particles of the or each other plurality by the coating. The coatings may differ from one plurality to the next in terms of coating thickness or composition, e.g. the ratio and/or identity of components. Multi-phasic release formulations would be particularly suitable for suffers of Crohn's disease affecting different regions along the intestine, including the ileum and/or the colon.

Medical Applications

The present invention provides a pharmaceutical composition according to the invention for use in therapy. It also provides a method of treating or preventing a disease or condition in a subject, especially a human subject, which comprises administering to the subject via the lower gastro-intestinal tract, especially the ileum and/or the colon a pharmaceutical composition which comprises as active ingredient a peptide, together with an amino acid, dipeptide or a combination thereof. Preferably, the compositions are adapted for administration via the oral or rectal route. Although the invention finds utility in the treatment of diseases of the lower gastro-intestinal tract, especially the ileum and/or the colon, it also has application as a portal for entry of a peptide into the systemic circulation by absorption from the lower gastro-intestinal tract, especially the ileum and/or the colon, and hence finds utility in the treatment of a wide range of diseases and conditions. It may for example find utility in the treatment or prevention of autoimmune diseases.

The invention finds particular utility in the treatment or prevention, including maintenance of remission or prevention of relapse, of a disease or condition of the ileum and/or the colon, especially the colon, for example inflammatory bowel disease (including ulcerative colitis and Crohn's disease), IBS, constipation, diarrhoea, infections, or cancer, and the invention therefore further provides the use of an amino acid, dipeptide or a combination thereof, in a method of manufacture of a medicament comprising a peptide for the treatment and/or prevention of one or more of these conditions. In these medicaments the amino acid, dipeptide or a combination thereof act to stabilise the peptide active ingredient.

The treatment and/or prevention of IBD is of particular importance.

The invention is illustrated by the following Examples which refer to the figures listed below:

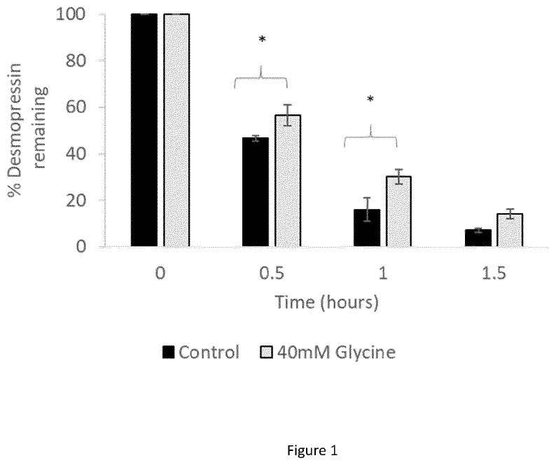

FIG. 1 shows the stabilisation of Desmopressin by glycine in human colon fluid

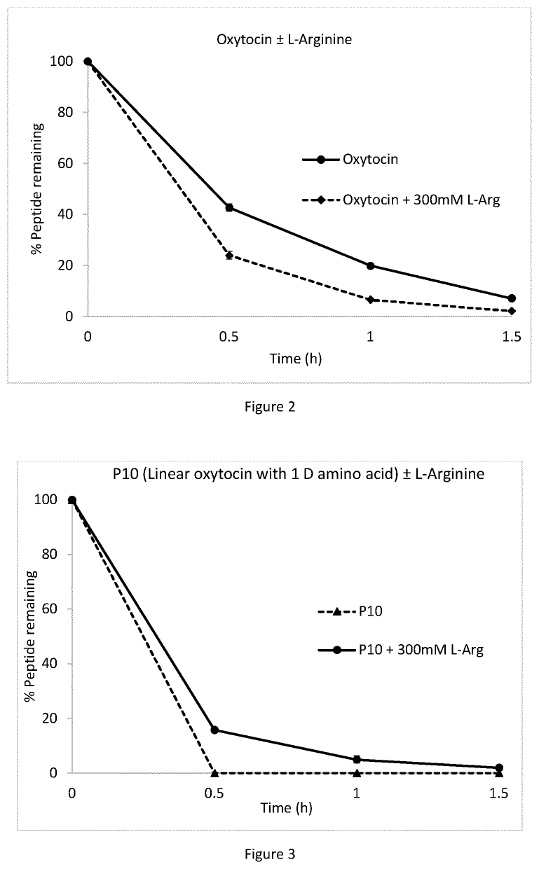

FIG. 2 shows the stabilising effect of L-Arginine on Oxytocin in human colon fluid

FIG. 3 shows the stabilising effect of L-Arginine on a Linear Oxytocin variant which has one D amino acid in human colon fluid.

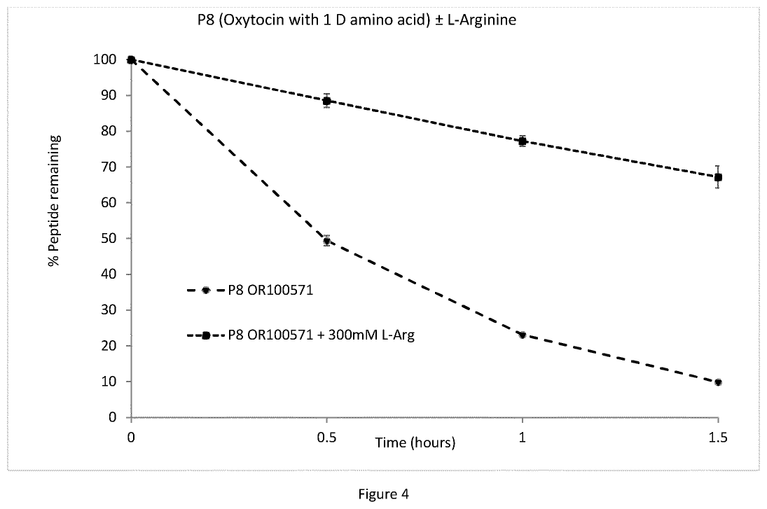

FIG. 4 shows the stabilising effect of L-Arginine on an Oxytocin variant which has one D amino acid in human colon fluid.

### MATERIALS AND METHODS

Human Colon Model

A human colonic model based on a mixed faecal inoculum was used to mimic the luminal environment of the human large intestine. An anaerobic workstation (Electrotek 500TG™ workstation, Electrotek, West Yorkshire, UK) maintained at 37° C. and 70% relative air humidity was used to set up the model. The fecal material was transferred in the anaerobic workstation and diluted with freshly prepared basal medium to obtain 20% w/w slurry by homogenization. The basal media provides nutrients and growth factors to the microbiota allowing viability for up to 24 hours. The homogenized bacterial media was sieved through an open mesh fabric (SefarNitex™, pore size 350 μm) to remove any nonhomogeneous fibrous material. The pH was maintained at approximately 7 to mimic the colonic luminal pH of the human.

Desmopressin Incubation Studies

Desmopressin stock solution was prepared in HPLC water at 2 mg/ml without or with single L-amino acids, combinations of L-amino acids or di-peptides at 10 and/or 20 and/or 40 mM concentrations and added to 25% w/w human faecal slurry; to obtain an incubation concentration of 1 mg/ml and 12.5% w/w faecal slurry. Samples were withdrawn at appropriate time points and added to HPLC grade methanol in a ratio of 1:1. The samples were centrifuged at 10.0×g for 10 minutes at 4° C. and the supernatant was analysed by Reversed Phase-HPLC (RP-HPLC).

RP-HPLC

Sample analysis was performed using a high performance liquid chromatography (HPLC) system (Agilent Technologies, 1260 Infinity II Series™) equipped with a pump (model G7111A), autosampler (model G7129A) and a diode-array UV detector (model G7114A). A 150×4.6-mm, 5 μm, 100 Å (Phenomenex, UK) reversed phase chromatography column was used for sample separation using 0.1% TFA in water (A) and acetonitrile (B) as the mobile phase for elution, at a flow rate of 1 ml/min. The gradient was set up with 17% of B at 0-4 minutes, 30% of B at 20 minutes and 17% of B at 20.01-23 minutes. The analysis was operated at room temperature and UV detection wavelength was set at 220 nm. Each sample was run for 23 minutes to allow complete elution of the sample without overlapping matrix interferences. The retention time for desmopressin was 13.7 minutes.

### Example 1. Stability of the Therapeutic Cyclic Peptide Desmopressin in the Presence of Amino Acids

Colon stability was assessed using the Human Colon model with the amount of intact desmopressin remaining at each time point assessed by RP-HPLC as described in the Methods section.

The results are shown in the following Table.

| | | % | % | % | |
|---|---|---|---|---|---|
| | | Desmopressin | Desmopressin | Desmopressin | |
| | | remaining after | remaining after | remaining | P value |
| | Concentration of | 0 hours | 0.5 hours | after 1 hours | (vs |
| Drug concentration | excipient | (T = 0 h) | (T = 0.5 h) | (T = 1 h) | control) |
| Desmopressin | — | 100 | — | 22.6 ± 2.7 | — |
| 0.1 mg/ml | | | | | |
| (control) | | | | | |
| Desmopressin | 20 mM L- | 100 | — | 35.6 ± 2.8 | <0.05 |
| 0.1 mg/ml | Phenylalanine | | | | |
| Desmopressin | 20 mM L- | 100 | — | 28.8 ± 1.2 | <0.05 |
| 0.1 mg/ml | Tyrosine | | | | |
| Desmopressin | — | 100 | 71.1 ± 4.6 | 37.7 ± 1.9 | >0.05 |
| 1 mg/ml | | | | | |
| Desmopressin | 10 mM L- | 100 | 69.9 ± 2.9 | 38.3 ± 0.82 | >0.05 |
| 1 mg/ml | Phenylalanine | | | | |
| Desmopressin | 10 mM L- | 100 | 71.4 ± 0.13 | 38.2 ± 1.9 | >0.05 |
| 1 mg/ml | Tyrosine | | | | |
| Desmopressin | 10 mM Tyr-Phe | 100 | 72.4 ± 2.6 | 40.7 ± 1.3 | >0.05 |
| 1 mg/ml | | | | | |
| Desmopressin | 10 mM Glycine | 100 | 72.4 ± 2.0 | 40.7 ± 0.73 | >0.05 |
| 1 mg/ml | | | | | |
| Desmopressin | — | 100 | 46.8 ± 1.2 | 15.9 ± 5.1 | — |
| 1 mg/ml (control) | | | | | |
| Desmopressin | 20 mM Glycine | 100 | 53.0 ± 2.8 | 26.8 ± 2.4 | <0.05 |
| 1 mg/ml | | | | | at T = 1 |
| Desmopressin | — | 100 | 38.3 ± 6.4 | 11.5 ± 2.7 | — |
| 1 mg/ml (control) | | | | | |
| Desmopressin | 20 mM L- | 100 | 42.1 ± 0.13 | 14.6 ± 2.6 | >0.05 |
| 1 mg/ml | Phenylalanine | | | | |
| Desmopressin | 20 mM L- | 100 | 37.9 ± 0.8 | 14.7 ± 2.8 | >0.05 |
| 1 mg/ml | Tyrosine | | | | |
| Desmopressin | 20 mM Tyr-Phe | 100 | 45 ± 5.4 | 16.7 ± 2.5 | >0.05 |
| 1 mg/ml | | | | | |
| Desmopressin | — | 100 | 46.8 ± 1.2 | 15.9 ± 5.1 | — |
| 1 mg/ml (control) | | | | | |
| Desmopressin | 40 mM Glycine | 100 | 56.6 ± 4.4 | 30.2 ± 3.1 | <0.05 |
| 1 mg/ml | | | | | at T = 0.5 |
| | | | | | and 1 |
| Desmopressin | 40 mM L- | 100 | 53.9 ± 3.7 | 25.0 ± 1.5 | <0.05 |
| 1 mg/ml | Phenylalanine | | | | at T = 1 |
| Desmopressin | 40 mM Tyr-Phe | 100 | 47.3 ± 2.7 | 24.4 ± 1.0 | <0.05 |
| 1 mg/ml | | | | | at T = 1 |
| Desmopressin | — | 100 | 72.8 ± 4.7 | 38.6 ± 2.0 | — |
| 1 mg/ml (control) | | | | | |
| Desmopressin | 40 mM L- | 100 | 68.7 ± 2.1 | 37.8 ± 0.6 | >0.05 |
| 1 mg/ml | Tyrosine | | | | |
| Desmopressin | — | 100 | 55.1 ± 4.7 | 27.1 ± 1.5 | — |
| 1 mg/ml (control) | | | | | |
| Desmopressin | 40 mM Glycine + | 100 | 61.7 ± 2.6 | 38.4 ± 2.6 | <0.05 |
| 1 mg/ml | 40 mM L- | | | | at T = 1 |
| | Phenylalanine | | | | |
| Desmopressin | 40 mM Gly-Phe | 100 | 60.5 ± 2.2 | 36.4 ± 1.5 | <0.05 |
| 1 mg/ml | | | | | at T = 1 |

The results show that the cyclic peptide desmopressin can be stabilised in the colonic lumen, as assayed in the Colonic Model in the presence of the amino acids glycine, phenylalanine or tyrosine, or combinations of these amino acids.

The stabilising effect of Glycine is illustrated in FIG. 1.

### Example 2. Stability of the Therapeutic Cyclic Peptide Oxytocin, Oxytocin with 1 D-Amino Acid and a Linear Form of Oxytocin in the Presence of Amino Acid L-Arginine in Human Colonic Fluid

Colon stability was assessed using the Human Colon model with the amount of intact oxytocin, oxytocin with 1 D-amino acid and linear oxytocin remaining at each time point assessed by RP-HPLC as described in the Methods section.

The results are shown in the following Table.

| | | % Peptide | % Peptide | % Peptide | P value |
|---|---|---|---|---|---|
| | Excipient | remaining at 0 | remaining at 0.5 | remaining at 1 | (vs |
| Drug concentration | concentration | hours (T = 0 h) | hours (T = 0.5 h) | hours (T = 1 h) | control) |
| Oxytocin 1 mg/ml | — | 100 | 24 ± 1.53 | 6.6 ± 0.25 | — |
| (control) | | | | | |
| Oxytocin 1 mg/ml | 300 mM L- | 100 | 42.7 ± 1.41 | 19.8 ± 0.92 | <0.001 |
| | arginine | | | | |
| Oxytocin 1 mg/ml | — | 100 | 49.4 ± 1.43 | 23.1 ± 0.19 | — |
| with 1 D-amino acid | | | | | |
| (control) | | | | | |
| Oxytocin 1 mg/ml | 300 mM L- | 100 | 88.5 ± 1.88 | 77.2 ± 1.48 | <0.001 |
| with 1 D-amino acid | arginine | | | | |
| Linear Oxytocin | — | 100 | 0 | 0 | |
| 1 mg/ml with 1 D- | | | | | |
| amino acid (control) | | | | | |
| Linear Oxytocin | 300 mM L- | 100 | 15.8 ± 0.46 | 5 ± 1.27 | <0.001 |
| 1 mg/ml with 1 D- | arginine | | | | at 0.5 h. |
| amino acid | | | | | P = 0.003 |
| | | | | | at 1 h |

The results, illustrated in FIGS. 2, 3 and 4, show that the cyclic peptide oxytocin, a variant with 1 D-amino acid and a linear version can be stabilised in the human colonic lumen, as assayed in the Colonic Model in the presence of the amino acid L-arginine.

1.-18. (canceled)

19. A pharmaceutical composition comprising (a) as active ingredient a peptide and (b) an amino acid, dipeptide or a combination thereof

20. The pharmaceutical composition of claim 19, wherein the active ingredient is a cyclic peptide.

21. The pharmaceutical composition of claim 19, wherein the pharmaceutical composition is a liquid, solid or semi-solid form suitable for oral or rectal administration.

22. The pharmaceutical composition of claim 19, wherein the pharmaceutical composition is a solid form suitable for oral administration, and wherein the pharmaceutical composition comprises an enteric coating.

23. The pharmaceutical composition of claim 19, wherein the pharmaceutical composition is adapted for selective release of the peptide in the lower gastro-intestinal tract.

24. The pharmaceutical composition of claim 19, wherein the pharmaceutical composition comprises a solid dosage form with a core and a coating for the core, the core comprising the active ingredient, and the amino acid, dipeptide or a combination thereof and the coating comprising a mixture of a digestible polysaccharide and a film-forming material which has a solubility threshold at pH 6.0 or above.

25. The pharmaceutical composition of claim 24, wherein the digestible polysaccharide is selected from the group consisting of starch; amylose; amylopectin; chitosan; chondroitin sulfate; cyclodextrin; dextran; pullulan; carrageenan; scleroglucan; chitin; curdulan and levan;

26. The pharmaceutical composition of claim 24, wherein the film-forming material is an acrylate polymer, a cellulose polymer or a polyvinyl-based polymer.

27. The pharmaceutical composition of claim 26, wherein the film-forming material is selected from cellulose acetate phthalate; cellulose acetate trimellitate; hydropropylmethylcellulose acetate succinate; and polyvinyl acetate phthalate.

28. The pharmaceutical composition of claim 19, wherein the peptide comprises 1, 2, 3, 4, or 5 D amino acids.

29. An orally administrable pharmaceutical composition comprising the pharmaceutical composition of claim 19.

30. A solid dosage form for oral administration, wherein the solid dose comprises a core comprising the pharmaceutical composition of claim 19.

31. A rectally administrable pharmaceutical composition comprising the pharmaceutical composition of claim 19.

32. An enema formulation comprising the pharmaceutical composition of claim 19.

33. A method of treating or preventing a disease or condition in a subject, wherein the method comprises administering to the subject via the ileum and/or the colon the pharmaceutical composition of claim 19.

34. The method of claim 33, wherein the disease or condition is inflammatory bowel disease; irritable bowel syndrome; constipation; diarrhoea; infection; autoimmune disease or cancer.

35. The method of claim 33, wherein the active ingredient is a cyclic peptide.

36. The method of claim 33, wherein the peptide comprises 1, 2, 3, 4, or 5 D amino acids.

37. A method of stabilizing a peptide in the presence of intestinal fluid, which comprises contacting the peptide with an amino acid, dipeptide or a combination thereof.

38. The method of claim 37, wherein the peptide is a cyclic peptide.

39. The method of claim 37, wherein the peptide comprises 1, 2, 3, 4, or 5 D amino acids.


SEQUENCE LISTING (1 sequence)

SEQ ID NO: 1 - protein, 8 aa, Homo sapiens
CYIQNPLG